(19) United States
(12) Patent Application Publication
(10) Pub. No.: US 2026/0078110 A1
(43) Pub. Date: Mar. 19, 2026

(54) SESTERTERPENOID COMPOUNDS AND EXTRACT L01 OF LEUCOSCEPTRUM CANUM, AND USE THEREOF IN TREATMENT OF PSORIASIS

(71) Applicant: Kunming Institute of Botany, CAS, Panlong District, Kunming, Yunnan (CN)

(72) Inventors: Shenghong LI, Panlong District, Kunming, Yunnan (CN); Kai GUO, Panlong District, Kunming, Yunnan (CN); Tingting ZHOU, Panlong District, Kunming, Yunnan (CN); Yanchun LIU, Panlong District, Kunming, Yunnan (CN); Yan LIU, Panlong District, Kunming, Yunnan (CN)

(21) Appl. No.: 18/850,204

(22) PCT Filed: May 13, 2024
(86) PCT No.: PCT/CN2024/092859

§ 371 Date: Sep. 24, 2024

(30) Foreign Application Priority Data
May 17, 2023 (CN) 202310552734.X

(51) Int. Cl. C07D 407/06; A61K 9/20; A61K 9/48; A61K 31/341; A61K 31/352; A61K 31/365; A61K 36/53; A61P 17/06; A61P 29/00; A61P 37/06; C07D 307/46; C07D 307/58; C07D 311/94

(52) U.S. Cl. CPC C07D 407/06; A61K 9/2018; A61K 9/2059; A61K 9/4858; A61K 31/341; A61K 31/352; A61K 31/365; A61K 36/53; A61P 17/06; A61P 29/00; A61P 37/06; C07D 307/46; C07D 307/58; C07D 311/94; A61K 2236/15; A61K 2236/17; A61K 2236/35; A61K 2236/39; A61K 2236/51; A61K 2236/55

(57) ABSTRACT

Sesterterpenoid compounds and an extract L01 of Leucosceptrum canum, and use thereof in treatment of psoriasis are provided, belonging to the technical field of natural medicinal chemistry. The sesterterpenoid compound of the Leucosceptrum canum has a structure shown in any one of Formulas 9 to 12, which shows a highly rare backbone type, exhibits anti-inflammatory and immunosuppressive activities, and can be used in preparation of an anti-inflammatory drug and an immunosuppressive drug. A Leucosceptrum canum extract L01 having compounds 1 to 12 as characteristic ingredients, and use thereof in preparation of an anti-inflammatory drug and an immunosuppressive drug are further provided.

The present application is a national phase of International Application No. PCT/CN2024/092859 entitled “SESTERTERPENOID COMPOUNDS AND EXTRACT L01 OF Leucosceptrum canum, AND USE THEREOF IN TREATMENT OF PSORIASIS” filed on May 13, 2024, which claims priority to Chinese Patent Application No. 202310552734.X filed to the China National Intellectual Property Administration (CNIPA) on May 17, 2023 and entitled “SESTERTERPENOID COMPOUNDS AND EXTRACT L01 OF Leucosceptrum canum, AND USE THEREOF IN PHARMACY”, and both of which are incorporated herein by reference in their entireties.

### TECHNICAL FIELD

The present disclosure belongs to the technical field of natural medicinal chemistry, and specifically relates to sesterterpenoid compounds and an extract L01 of Leucosceptrum canum, and use thereof in treatment of psoriasis.

### BACKGROUND

Autoimmune diseases, including such as psoriasis, rheumatoid arthritis, systemic lupus erythematosus, inflammatory bowel disease, and multiple sclerosis are a type of disease caused by damage to autologous tissues resulting from the body's immune system producing local or systemic abnormal immune responses against self-antigens, and include psoriasis, rheumatoid arthritis, systemic lupus erythematosus, inflammatory bowel disease, and multiple sclerosis. Currently, there are more than 80 autoimmune diseases reported, with diverse clinical manifestations and increasing incidence, which have a serious impact on human health.

Psoriasis, also called “cattle-skin lichen”, is a chronic autoimmune disease with a global prevalence of 2% to 3%. The psoriasis is divided into plaque psoriasis, guttate psoriasis, pustular psoriasis, and crythrodermic psoriasis, of which the plaque psoriasis is the most common type, with clinical manifestations mostly of erythema and desquamation, and can occur all over the body. Psoriasis patients generally suffer from psoriatic arthritis, lymph cancer, cardiovascular disease, hypertension, diabetes, and depression. At present, immunosuppressive drugs are mainly used in the clinical treatment of autoimmune diseases. Although there are many immunosuppressive drugs with desirable efficacy for clinical use, there are still various problems such as large toxic side effects, unsatisfactory efficacy, and high prices. As a result, there is an urgent need for research and development of new immunosuppressive drugs.

Natural products play an important role in the treatment of autoimmune diseases due to their unique mechanism of action as well as minimal toxicity and side effects. Many natural products derived from Chinese herbal medicines have significant anti-inflammatory and immunosuppressive effects, especially plant-derived terpenoid compounds show remarkable anti-inflammatory and immunosuppressive activities and are an important source for discovering novel natural anti-inflammatory and immunosuppressive drugs.

Leucosceptrum canum, a monospecies of plant in the genus Leucosceptrum of the family Lamiaceae, is a perennial shrub or small tree. Leaves and bark of the Leucosceptrum canum have a high medicinal value, which are used by the people in Yunnan Province to relieve tendons, stop bleeding, and reduce inflammation, thereby treating fever, stomach pain, traumatic bleeding, closed fractures, impetigo and other diseases. So far, some sesterterpenoid-based chemical components have been found in the Leucosceptrum canum, but there are no reports on the anti-inflammatory and immunosuppressive activities of sesterterpenoids in the Leucosceptrum canum.

### SUMMARY

In view of this, an objective of the present disclosure is to provide sesterterpenoid compounds and an extract L01 of Leucosceptrum canum, and use thereof in treatment of psoriasis.

To achieve the above objective, the present disclosure provides the following technical solutions:

The present disclosure provides a sesterterpenoid compound shown in any of the structural formulas 9 to 12 as follows:

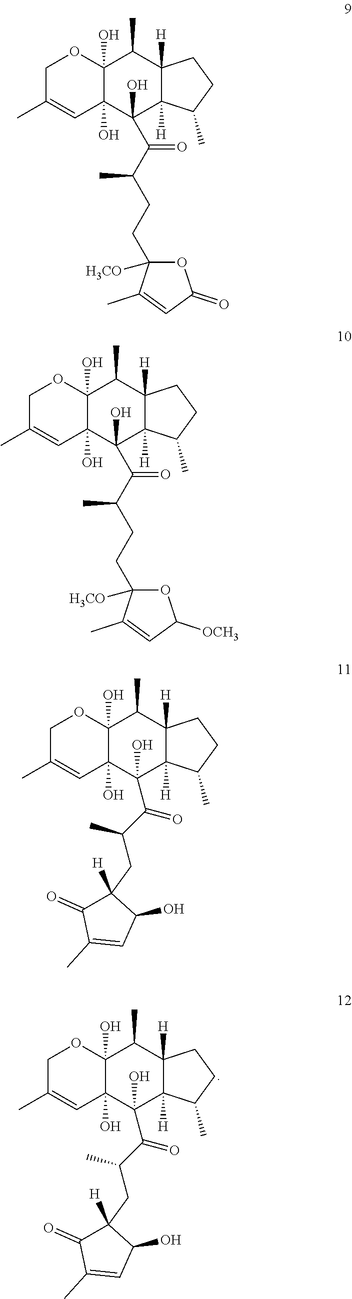

The present disclosure further provides a method for preparing the sesterterpenoid compounds 9 to 12, including the following steps:
(1) extracting a Leucosceptrum canum with an organic solvent to obtain an extract solution;
(2) vacuum distilling the extract solution and concentrating in sequence to obtain an extractum; and
(3) dissolving the extractum, followed by separating the extractum by column chromatography and high-performance liquid chromatography (HPLC) in sequence to obtain the sesterterpenoid compounds.

In the present disclosure, the column chromatography includes silica gel column chromatography, middle chromatogram isolated (MCI) column chromatography or reverse phase C₁₈ column chromatography, and dextran gel column chromatography; and the HPLC separation is conducted by preparative HPLC or semi-preparative HPLC.

In the present disclosure, a Leucosceptrum canum is extracted with an organic solvent to obtain an extract solution. Before the extraction, the Leucosceptrum canum is preferably crushed into 30 mesh size. The Leucosceptrum canum can be collected from an overground part or any tissue of Leucosceptrum canum, such as leaves, flowers, or stems of the Leucosceptrum canum for shade drying; there is no special requirement on moisture content of the Leucosceptrum canum, and both fresh and dried Leucosceptrum canum are acceptable.

In the present disclosure, the organic solvent preferably includes one or more of methanol, ethanol, acetone, chloroform, dichloromethane (DCM), and petroleum ether; 5 L of the organic solvent is preferably added to per kilogram of a dry weight of the Leucosceptrum canum. The extraction is preferably cold soaking extraction or hot reflux extraction. The cold soaking extraction is conducted at preferably a room temperature.

The cold soaking extraction is conducted preferably 2 to 5 times, more preferably 3 to 4 times; each extraction is conducted for preferably 24 h to 48 h, more preferably 36 h to 48 h; after each cold soaking extraction is completed, an obtained extraction system is filtered and a filtrate after each filtration is combined. The hot reflux extraction is conducted at preferably 40° C. to 50° C., more preferably 45° C. to 50° C. for preferably 4 h to 6 h, more preferably 4 h to 5 h.

In the present disclosure, the extract is vacuum distilled for concentration to obtain an extractum. The vacuum distillation is conducted at preferably 35° C. to 45° C. for preferably 60 min to 100 min; the concentration factor by vacuum distillation is preferably 50 times. After the vacuum distillation is completed, the organic solvent in the extract solution is removed while the extract solution is concentrated.

In the present disclosure, the extractum is dissolved, and column chromatography separation and HPLC separation are conducted in sequence to obtain the sesterterpenoid compounds. The extractum is preferably dissolved in chloroform, and a mass of the chloroform is 1.5 to 3 times that of the extractum. The column chromatography includes preferably silica gel column chromatography, MCI column chromatography or reverse phase C₁₈ column chromatography, and dextran gel column chromatography. A column loading process of the silica gel column chromatography includes preferably: loading with the silica gel of 200 mesh size to 300 mesh size; mixing a resulting dissolved extractum with 1 to 1.5 times a mass of the silica gel of 200 mesh size to 300 mesh size, and subjecting a resulting mixed sample to air drying, grinding, and sieving, and then transferring a resulting sieved sample into a silica gel column. The silica gel column chromatography preferably adopts gradient elution, and a mobile phase for the gradient elution is preferably a solvent mixture of a solvent A and a solvent B at a volume ratio ranging from 1:0 to 0:1; and the solvent A is preferably any one selected from the group consisting of DCM, chloroform, and petroleum ether, and the solvent B is preferably any one selected from the group consisting of DCM, chloroform, acetone, ethyl acetate, and methanol. Preferably, fractions obtained by the gradient elution are subjected to vacuum concentration and TLC detection in sequence, and then same fractions are combined. The gradient elution of the silica gel column chromatography preferably includes: conducting gradient elution with PE (petroleum ether):CH₂Cl₂ (1:0, 1:1, 0:1, v/v) and CH₂Cl₂:Me₂CO (9:1, 4:1, 1:1) in sequence, and combining obtained fractions after TLC detection to obtain a total of 6 fractions to be separated, including Fr.1 (PE), Fr.2 (PE:CH₂Cl₂=1:1), Fr.3 (CH₂Cl₂), Fr.4 (CH₂Cl₂:Me₂CO=9:1), Fr.5 (CH₂Cl₂:Me₂CO=4:1), and Fr.6 (CH₂Cl₂:Me₂CO=1:1).

In the present disclosure, the fraction Fr.4 is subjected to the MCI column chromatography or the reverse phase C₁₈ column chromatography, and an obtained fraction Fr.4-2 is subjected to the dextran gel column chromatography. In a specific example, the dextran gel has a model number of LH-20. A mobile phase for the dextran gel column chromatography is preferably a solvent mixture of DCM and methanol at a volume ratio of 1:1; after the dextran gel column chromatography, 4 sub-fractions are obtained, namely Fr.4-2-1, Fr.4-2-2, Fr.4-2-3, and Fr.4-2-4.

In the present disclosure, after the column chromatography is completed, the sub-fractions Fr.4-2-1, Fr.4-2-2, Fr.4-2-3, and Fr.4-2-4 are subjected to HPLC separation to obtain the sesterterpenoid compounds. The HPLC separation adopts preparative HPLC or semi-preparative HPLC; chromatographic conditions of the HPLC separation preferably include: a chromatographic column is selected from the group consisting of a C₁₈ chromatographic column, a C₈ chromatographic column, a phenyl chromatographic column, and a silica gel chromatographic column, and has a column temperature of 35° C.; gradient elution is conducted with a mobile phase being a solvent mixture of acetonitrile and water or a solvent mixture of methanol and water at a volume ratio of 100:0 to 5:95; and a flow rate is 3 mL/min. In a specific example, the HPLC separation preferably includes: using an Agilent 1200 semi-preparative liquid chromatograph and a ZorbaxSB-C₁₈ column (5 μm, 9.4×250 mm, 3 mL/min), a column temperature of 35° C.; under an elution condition of MeCN—H₂O (75:25, v/v),

the compound 3 (t_{R}14.5 min), compound 4 (t_{R}17.2 min), and compound 6 (t_{R}21.4 min) are obtained from the fraction Fr.4-2-1; under an elution condition of MeCN—H₂O (70:30, v/v), the compound 1 (t_{R}11.6 min), compound 8 (t_{R}13.5 min), and compound 10 (t_{R}14.8 min) are obtained from the fraction Fr.4-2-2; under an elution condition of MeCN—H₂O (80:20, v/v), the compound 7 (t_{R}9.7 min), compound 9 (t_{R}10.5 min), and compound 12 (t_{R}13.8 min) are obtained from the fraction Fr.4-2-3; under an elution condition of MeCN—H₂O (60:40, v/v), the compound 11 (t_{R}11.5 min), compound 2 (t_{R}14.4 min), and compound 5 (t_{R}28.6 min) are obtained from the fraction Fr.4-2-4.

The present disclosure further provides the Leucosceptrum canum extract L01 and a preparation method thereof, where the preparation method includes the following steps:
(1) mixing a crushed Leucosceptrum canum an organic solvent to allow an ultrasonic treatment, and then collecting a resulting extract; where the organic solvent is one or more selected from the group consisting of methanol, ethanol, acetone, chloroform, DCM, and petroleum ether; an overground part of the crushed Leucosceptrum canum and the organic solvent are at a material-to-liquid ratio of 1:(3-8); and the ultrasonic treatment is conducted for 10 min to 40 min;
(2) subjecting the extract solution to concentration to obtain a crude extract; where the concentration is conducted by rotary evaporation at 30° C. to 60° C.; and
(3) subjecting the crude extract to elution by silica gel column chromatography, collecting a resulting acetone elution phase, and conducting concentration to obtain the Leucosceptrum canum extract L01; where eluents in the elution by silica gel column chromatography are petroleum ether and acetone in sequence; and the concentration is conducted by rotary evaporation at 30° C. to 60° C.

Specifically, the preparation method of the Leucosceptrum canum extract L01 includes the following steps:
(1) collecting an overground part of Leucosceptrum canum to allow shade drying, crushing to 30 mesh size, fully mixing a crushed Leucosceptrum canum with 10 L of petroleum ether at room temperature, subjecting a resulting mixture to ultrasonic extraction three times for 30 min each time, conducting filtration, and combining obtained extracts to obtain a crude extract;
(2) subjecting the crude extract to concentration by a rotary evaporator at 45° C. to obtain an extractum; and
(3) dissolving the extractum in 0.5 L of chloroform, mixing with a silica gel of 200 mesh size to 300 mesh size, conducting air drying, grinding and sieving, loading with the silica gel filler of 200 mesh size to 300 mesh size to allow column chromatography, conducting elution with petroleum ether and acetone in sequence, and subjecting an obtained acetone eluate to concentration to obtain the Leucosceptrum canum extract L01.

The present disclosure further provides use of the sesterterpenoid compounds 1 to 12 and the extract L01 in preparation of an anti-inflammatory drug or an immunosuppressive drug, as well as in preparation of a T cell proliferation inhibitor or a cytokine inhibitor and an anti-psoriasis drug.

Further, the present disclosure also provides a pharmaceutical composition, including a pharmaceutically acceptable carrier and one or more selected from the group consisting of the sesterterpenoid compounds 1 to 12.

The present disclosure further provides a pharmaceutical composition, including a pharmaceutically acceptable carrier and the Leucosceptrum canum extract L01.

In the present disclosure, the compounds 1 to 12 or the extract L01 in the pharmaceutical composition have a mass percentage content of preferably 0.1% to 99%, more preferably 0.5% to 90%; and the pharmaceutically acceptable carrier in the pharmaceutical composition has a total mass percentage content of preferably 1% to 99.9%, more preferably 10% to 99.5%.

In the present disclosure, there is no particular limitation on the pharmaceutically acceptable carrier, and any pharmaceutically acceptable carrier well known in the art may be used, specifically, such as one or more selected from the group consisting of solid, semisolid, liquid diluent, filler and pharmaceutical product adjuvant. A dosage form of the pharmaceutical composition preferably is selected from the group consisting of a liquid preparation, a solid preparation, a spray, and an aerosol; where the liquid preparation is preferably selected from the group consisting of an injection, a suspension, an emulsion, a solution, and a syrup; and the solid preparation is preferably selected from the group consisting of a tablet, a capsule, a granule, and an instant granule.

In the present disclosure, there is no particular limitation on a preparation process of the pharmaceutical composition, and any preparation process well known in the art may be used.

In the present disclosure, the pharmaceutical composition and the extract L01 are preferably administrated by injection, oral administration, sublingual administration, or mucosal dialysis; the injection is preferably intravenous injection, intravenous drip, intramuscular injection, intraperitoneal injection, or subcutaneous injection.

In the present disclosure, the pharmaceutical composition and the extract L01 are preferably used in the form of a dosage per unit body weight. The dosage per unit of body weight is preferably 0.5 mg/kg to 500 mg/kg.

The present disclosure has the following beneficial effects by using the above technical solutions:

In the present disclosure, the sesterterpenoid compounds 1 to 12 prepared by the technical solutions herein significantly reduce secretion of an inflammatory cytokine IFN-γ in mouse T cells stimulated by CD3/CD28 monoclonal antibodies during in vitro experiments.

During the in vivo experiment on mice, the compound 6 and the Leucosceptrum canum extract L01 both have significantly reduced the levels of pro-inflammatory cytokines IFN-γ and IL-17A in the serum of psoriasis model mice, thereby significantly improving the clinical symptoms of psoriasis mice such as weight loss, back skin scales, and thickness, and reduced pathological damage to the mouse skin and systemic immune response. This provides choices for screening of safe and efficient immunosuppressants, and also provides effective strategies for the treatment of autoimmune diseases such as psoriasis.

### BRIEF DESCRIPTION OF THE DRAWINGS

FIG. 1 shows a structure of the sesterterpenoid compound 1 in the present disclosure by single-crystal X-ray diffraction;

FIG. 2 shows a structure of the sesterterpenoid compound 2 in the present disclosure by single-crystal X-ray diffraction;

FIG. 3 shows a structure of the sesterterpenoid compound 4 in the present disclosure by single-crystal X-ray diffraction;

FIG. 4 shows schematic structural diagram of the sesterterpenoid compounds 1 to 12 in the present disclosure;

FIG. 5 shows the back skin of mice in each group after being treated with the Leucosceptrum canum extract L01 in the present disclosure;

FIG. 6 shows H&E staining sections of the back skin of mice after being treated with the Leucosceptrum canum extract L01 in the present disclosure;

FIGS. 7A-7B are diagrams showing changes in the levels of cytokines IFN-γ and IL-17A in mouse serum after being treated with the Leucosceptrum canum extract L01 in the present disclosure;

FIG. 8 shows the back skin of mice in each group after being treated with the sesterterpenoid compound 6 in the present disclosure;

FIG. 9 shows H&E staining sections of the back skin of mice after being treated with the sesterterpenoid compound 6 in the present disclosure; and

FIGS. 10A-10B are diagrams showing changes in the levels of cytokines IFN-γ and IL-17A in mouse serum after being treated with the sesterterpenoid compound 6 in the present disclosure.

### DETAILED DESCRIPTION OF THE EMBODIMENTS

The sesterterpenoid compounds and the extract L01 of Leucosceptrum canum, and the use thereof in treatment of psoriasis provided by the present disclosure are further illustrated below with reference to the accompanying drawings using the examples of the present disclosure, but they should not be construed as limiting the scope of protection of the present disclosure.

### Example 1

Specifically, the extraction and separation steps of the sesterterpenoid compounds 1 to 12 are as follows:
(1) The overground parts of the Leucosceptrum canum were collected from Yunnan Province, shade-dried and crushed to 30 mesh size to obtain 10.0 kg of sample. The sample was soaked in 50 L of petroleum ether and extracted 3 times at room temperature, with each time for 48 h. The obtained extract solution was combined after filtration, and after removing the solvent by vacuum distillation, a total of 230 g extractum was obtained.
(2) The resulted extractum was dissolved in 2 L of chloroform, mixed with 253 g of silica gel (200-300 mesh size), allowed to air drying, ground and sieved, and loaded with 2.0 kg of silica gel (200-300 mesh size) to allow column chromatography. Gradient elution was conducted with petroleum ether:DCM (1:0, 1:1, 0:1, v/v) and chloroform:acetone (9:1, 4:1, 1:1) in sequence. The obtained fractions were subjected to vacuum concentration and detected by TLC. The same fractions confirmed by TLC were combined, then a total of 6 fractions to be separated were obtained, namely Fr.1 (petroleum ether), Fr.2 (petroleum ether:chloroform=1:1), Fr.3 (chloroform), Fr.4 (chloroform:acetone=9:1), Fr.5 (chloroform:acetone=4:1), and Fr.6 (chloroform:acetone=1:1).
(3) The Fr.4 (48 g) was subjected to MCI column chromatography and gradient elution with methanol:water (50%, 60%, 70%, 80%, 90%, and 100%, v/v) to obtain 4 sub-fractions Fr.4-1 to Fr.4-4.

The fraction Fr.4-2 was subjected to the dextran gel column chromatography and then eluted with DCM:methanol (1:1, v/v) to obtain 4 sub-fractions Fr.4-2-1 to Fr.4-2-4.

The fractions Fr.4-2-1 to Fr.4-2-4 were subjected to HPLC separation:elution was conducted using an Agilent 1200 semi-preparative liquid chromatograph and ZorbaxSB-C₁₈ column (5 μm, 9.4×250 mm, 3 mL/min), at a column temperature of 35° C. Under an elution condition of MeCN—H₂O (75:25, v/v), compound 3 (t_{R}14.5 min), compound 4 (t_{R}17.2 min), and compound 6 (t_{R}21.4 min) were obtained from the fraction Fr.4-2-1; under elution conditions of MeCN—H₂O (70:30, v/v), compound 1 (t_{R}11.6 min), compound 8 (t_{R}13.5 min), and compound 10 (t_{R}14.8 min) were obtained from the fraction Fr.4-2-2; under an elution condition of MeCN—H₂O (80:20, v/v),

compound 7 (t_{R}9.7 min), compound 9 (t_{R}10.5 min), and compound 12 (t_{R}13.8 min) were obtained from the fraction Fr.4-2-3; under an elution condition of MeCN—H₂O (60:40, v/v), compound 11 (t_{R}11.5 min), compound 2 (t_{R}14.4 min), and compound 5 (t_{R}28.6 min) were obtained from the fraction Fr.4-2-4.

The obtained mass of each compound was as follows: compound 1 (2.0 mg), compound 2 (3.2 mg), compound 3 (9.5 mg), compound 4 (3.5 mg), compound 5 (2.0 mg), compound 6 (35.0 mg), compound 7 (2.0 mg), compound 8 (4.0 mg), compound 9 (3.5 mg), compound 10 (13.0 mg), compound 11 (1.2 mg), and compound 12 (2.5 mg).

The physical and spectral data of compounds 1 to 12 were as follows:

Compound 1: white solid; specific rotation [α]_{D}²⁰+12.0 (c 0.30, MeOH); infrared IR (KBr) νₘₐₓ 3429, 2955, 2931, 2870, 1742, 1721, 1640, 1377, 1164 cm⁻¹; high resolution mass spectrum HRESIMS m/z 431.2443 [M-H]⁻; ¹HNMR and ¹³CNMR data were shown in Table 1.

Compound 2: white solid; specific rotation [α]_{D}²⁰+34.9 (c 0.10, MeOH); infrared IR (KBr) νₘₐₓ 3435, 2973, 2936, 2871, 1760, 1716, 1452, 1378, 1140 cm⁻¹; high resolution mass spectrum HRESIMS m/z 455.2405 [M+Na]⁺; ¹HNMR and ¹³CNMR data were shown in Table 1.

Compound 3: colorless oily liquid; specific rotation [α]_{D}²⁰+61.7 (c 0.10, MeOH); infrared IR (KBr) νₘₐₓ 3437, 2973, 2936, 2871, 1715, 1452, 1377, 1138, 1013 cm⁻¹; high resolution mass spectrum HRESIMS m/z 439.2459 [M+Na]⁺; ¹HNMR and ¹³CNMR data were shown in Table 1.

Compound 4: colorless oily liquid; specific rotation [α]_{D}²⁰+61.5 (c 0.13, MeOH); infrared IR (KBr) νₘₐₓ 3470, 2967, 2938, 2874, 1684, 1462, 1394, 1378, 1010 cm⁻¹; high resolution mass spectrum HRESIMS m/z 375.2141 [M+Na]⁺; ¹HNMR and ¹³CNMR data were shown in Table 1.

Compound 5: colorless oily liquid; specific rotation [α]_{D}²⁰+39.0 (c 0.06, MeOH); infrared IR (KBr) νₘₐₓ 3438, 2971, 2939, 2871, 1704, 1684, 1452, 1379, 1055 cm⁻¹; high resolution mass spectrum HRESIMS m/z 391.2091 [M+Na]⁺; ¹HNMR and ¹³CNMR data were shown in Table 2.

Compound 6: white solid; specific rotation [α]_{D}²⁰+38.0 (c 0.10, MeOH); infrared IR (KBr) νₘₐₓ 3429, 2956, 2874, 1766, 1661, 1457, 1380, 1166 cm⁻¹; high resolution mass spectrum HRESIMS m/z 455.2396 [M+Na]⁺; ¹HNMR and ¹³CNMR data were shown in Table 2.

Compound 7: colorless oily liquid; specific rotation [α]_{D}²⁰+60.8 (c 0.05, MeOH); infrared IR (KBr) νₘₐₓ 3435, 2937, 2870, 1712, 1674, 1451, 1378, 1144 cm⁻¹; high resolution mass spectrum HRESIMS m/z 469.2195 [M+Na]⁺; ¹HNMR and ¹³CNMR data were shown in Table 2.

Compound 8: colorless oily liquid; specific rotation [α]_{D}²⁰+60.7 (c 0.06, MeOH); infrared IR (KBr) νₘₐₓ 3436, 2970, 2871, 1715, 1687, 1452, 1379, 1170 cm⁻¹; high resolution mass spectrum HRESIMS m/z 471.2353 [M+Na]⁺; ¹HNMR and ¹³CNMR data were shown in Table 2.

Compound 9: colorless oily liquid; specific rotation [α]_{D}²⁰+29.3 (c 0.10, MeOH); infrared IR (KBr) νₘₐₓ 2969, 2939, 2871, 1770, 1685, 1451, 1380, 1168 cm⁻¹; high resolution mass spectrum HRESIMS m/z 501.2458 [M+Na]⁺; ¹HNMR and ¹³CNMR data were shown in Table 3.

Compound 10: colorless oily liquid; specific rotation [α]_{D}²⁰+60.7 (c 0.06, MeOH); infrared IR (KBr) νₘₐₓ 3451, 2969, 2939, 2871, 1683, 1449, 1380, 1371 cm⁻¹; high resolution mass spectrum HRESIMS m/z 517.2775 [M+Na]⁺; ¹HNMR and ¹³CNMR data were shown in Table 3.

Compound 11: colorless oily liquid; specific rotation [α]_{D}²⁰+32.0 (c 0.11, MeOH); infrared IR (KBr) νₘₐₓ 3446, 2960, 2833, 1730, 1690, 1422, 1381, 1200 cm⁻¹; high resolution mass spectrum HRESIMS m/z 471.2353 [M+Na]⁺; ¹HNMR and ¹³CNMR data were shown in Table 3.

Compound 12: colorless oily liquid; specific rotation [α]_{D}²⁰+45.5 (c 0.06, MeOH); infrared IR (KBr) νₘₐₓ 3433, 2966, 2835, 1718, 1688, 1441, 1377, 1175 cm⁻¹; high resolution mass spectrum HRESIMS m/z 471.2353 [M+Na]⁺; ¹HNMR and ¹³CNMR data were shown in Table 3.

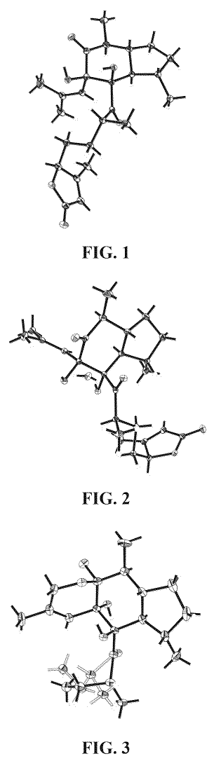

FIG. 1 to FIG. 3 show structures of the sesterterpenoid compounds 1, 2, and 4 by single-crystal X-ray diffraction.

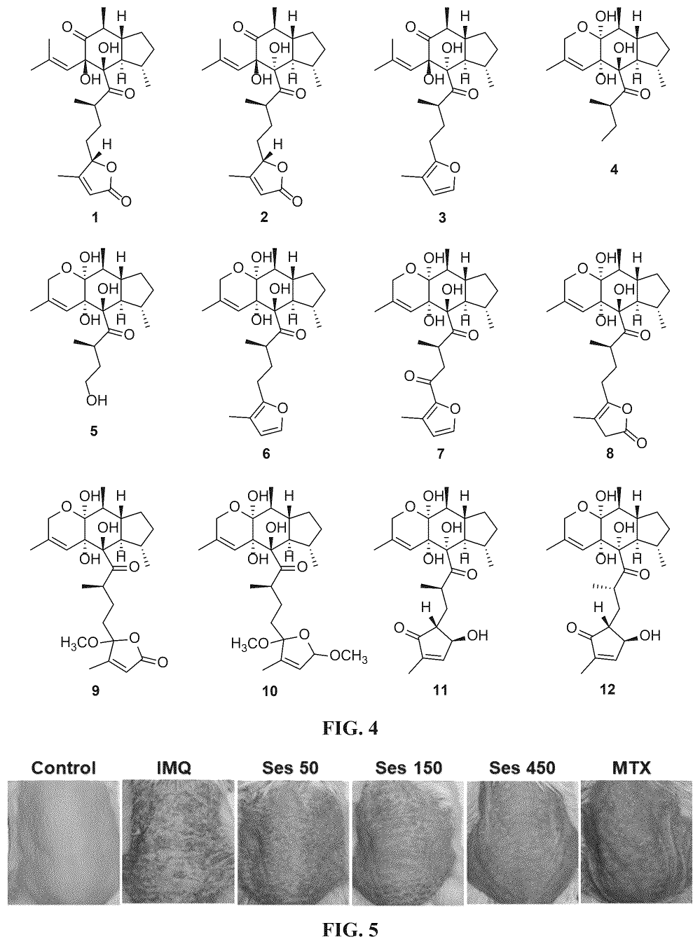

From the physical and spectral data of compounds 1 to 12, it was seen that compounds 1 to 12 conformed to the structures shown in FIG. 4.

TABLE 1
¹HNMR and ¹³CNMR data of compounds 1 to 4 (acetone-d₆, J in Hz)

| | 1 | | | | 2 | | | | 3 | | | | 4 | | | |
|---|---|---|---|---|---|---|---|---|---|---|---|---|---|---|---|---|
| no. | δ_{H} | | δ_{C} | | δ_{H} | | δ_{C} | | δ_{H} | | δ_{C} | | δ_{H} | | δ_{C} | |
| 1a | 1.52 | br s | 18.8 | q | 1.60 | d (1.3) | 19.0 | q | 1.60 | br s | 18.9 | q | 4.16 | d (16.0) | 64.9 | t |
| 1b | | | | | | | | | | | | | 4.02 | d (16.0) | | |
| 2 | | | 139.9 | s | | | 141.3 | s | | | 141.2 | s | | | 136.8 | s |
| 3 | 5.87 | br s | 121.9 | d | 5.68 | br s | 122.6 | d | 5.67 | br s | 122.6 | d | 4.97 | br s | 122.9 | d |
| 4 | | | 82.8 | s | | | 84.0 | s | | | 83.8 | s | | | 73.9 | s |
| 5 | | | 210.3 | s | | | 208.7 | s | | | 208.6 | s | | | 97.5 | s |
| 6 | 2.75 | m | 46.0 | d | 2.53 | m | 45.2 | d | 2.51 | m | 45.1 | d | 1.92 | m | 42.5 | d |
| 7 | 1.78 | m | 49.3 | d | 1.75 | m | 46.6 | d | 1.65 | m | 46.5 | d | 1.59 | m | 43.9 | d |
| 8a | 1.77 | m | 28.2 | t | 1.89 | m | 28.3 | t | 1.87 | m | 28.2 | t | 1.78 | m | 28.5 | t |
| 8b | 1.34 | m | | | 1.63 | m | | | 1.25 | m | | | 1.20 | m | | |
| 9a | 1.69 | m | 33.8 | t | 1.90 | m | 33.1 | t | 1.86 | m | 33.0 | t | 1.87 | m | 33.7 | t |
| 9b | 1.32 | m | | | 1.27 | m | | | 1.24 | m | | | 1.23 | m | | |
| 10 | 1.93 | m | 33.2 | d | 1.54 | m | 35.9 | d | 1.45 | m | 35.9 | t | 1.93 | m | 32.2 | d |
| 11 | 2.33 | m | 52.7 | d | 1.76 | m | 56.9 | d | 1.72 | m | 56.6 | d | 1.85 | m | 55.1 | d |
| 12 | | | 86.3 | s | | | 90.4 | s | | | 90.5 | s | | | 83.1 | s |
| 13 | | | 215.1 | s | | | 217.7 | s | | | 217.9 | s | | | 222.6 | s |
| 14 | 3.38 | m | 41.8 | d | 3.28 | m | 42.3 | d | 3.25 | m | 41.4 | d | 3.06 | m | 44.7 | d |
| 15a | 1.64 | m | 28.0 | t | 1.62 | m | 26.7 | t | 1.81 | m | 31.0 | t | 1.89 | m | 25.6 | t |
| 15b | 1.33 | m | | | 1.22 | m | | | 1.45 | m | | | 1.24 | m | | |
| 16a | 1.82 | m | 30.2 | t | 2.04 | m | 30.1 | t | 2.57 | m | 23.8 | t | 0.90 | t (7.5) | 12.0 | q |
| 16b | 1.32 | m | | | 1.50 | m | | | | | | | | | | |
| 17 | 4.98 | m | 84.3 | d | 4.95 | m | 84.8 | d | | | 151.3 | s | | | | |
| 18 | | | 170.7 | s | | | 170.0 | s | | | 114.5 | s | | | | |
| 19 | 5.79 | m | 116.7 | d | 5.81 | m | 117.3 | d | 6.17 | d (1.9) | 113.5 | d | | | | |
| 20 | | | 173.2 | s | | | 173.1 | s | 7.27 | d (1.9) | 140.8 | d | | | | |
| 21 | 1.71 | br s | 27.5 | q | 1.71 | d (1.5) | 27.9 | q | 1.71 | br s | 27.9 | q | 1.66 | br s | 17.7 | q |
| 22 | 1.01 | d (6.4) | 12.6 | q | 1.03 | d (6.5) | 12.9 | q | 1.02 | d (6.4) | 12.9 | q | 0.99 | d (6.8) | 11.4 | q |
| 23 | 0.86 | d (6.0) | 21.1 | q | 1.23 | (d, 6.5) | 21.5 | q | 1.16 | d (6.4) | 21.4 | q | 0.78 | d (6.0) | 21.6 | q |
| 24 | 1.10 | d (7.2) | 18.4 | q | 1.06 | (d, 6.9) | 16.5 | q | 1.09 | d (6.8) | 16.1 | q | 1.01 | d (6.8) | 14.7 | q |
| 25 | 2.04 | br s | 13.6 | q | 2.09 | br s | 13.7 | q | 1.94 | br s | 9.8 | q | | | | |
| 4-OH | 4.94 | s | | | 4.21 | s | | | 4.18 | s | | | 4.74 | s | | |
| 5-OH | | | | | | | | | | | | | 4.28 | s | | |
| 12-OH | 4.78 | s | | | 4.46 | s | | | 4.36 | s | | | 3.90 | s | | |

TABLE 2
¹HNMR and ¹³CNMR data of compounds 5 to 8 (acetone-d₆, J in Hz)

| | 5 | | | | 6 | | | | 7 | | | | 8 | | | |
|---|---|---|---|---|---|---|---|---|---|---|---|---|---|---|---|---|
| no. | δ_{H} | | δ_{C} | | δ_{H} | | δ_{C} | | δ_{H} (J in Hz) | | δ_{C} | | δ_{H} (J in Hz) | | δ_{C} | |
| 1a | 4.14 | d (16.0) | 64.9 | t | 4.17 | d (16.0) | 65.0 | t | 4.15 | (d, 16.0) | 65.0 | t | 4.15 | (d, 16.0) | 65.0 | t |
| 1b | 4.01 | d (16.0) | | | 4.02 | d (16.0) | | | 4.01 | (d, 16.0) | | | 4.01 | (d, 16.0) | | |
| 2 | | | 136.9 | s | | | 137.0 | s | | | 137.1 | s | | | 137.2 | s |
| 3 | 5.05 | m | 123.0 | d | 4.95 | d (1.4) | 122.9 | d | 5.28 | m | 123.1 | d | 4.95 | m | 122.8 | d |
| 4 | | | 73.7 | s | | | 73.9 | s | | | 73.9 | s | | | 73.9 | s |
| 5 | | | 97.6 | s | | | 97.6 | s | | | 97.6 | s | | | 97.6 | s |
| 6 | 1.90 | m | 42.6 | d | 1.95 | m | 42.6 | d | 1.91 | m | 42.6 | d | 1.91 | m | 42.5 | d |
| 7 | 1.58 | m | 43.9 | d | 1.62 | m | 44.0 | d | 1.57 | m | 43.8 | d | 1.57 | m | 43.9 | d |
| 8a | 1.74 | m | 28.5 | t | 1.77 | m | 28.6 | t | 1.76 | m | 28.5 | t | 1.87 | m | 28.5 | t |
| 8b | 1.21 | m | | | 1.20 | m | | | 1.20 | m | | | 1.20 | m | | |
| 9a | 1.87 | m | 33.7 | t | 1.91 | m | 33.8 | t | 1.75 | m | 33.6 | t | 1.86 | m | 33.7 | t |
| 9b | 1.22 | m | | | 1.25 | m | | | 1.21 | m | | | 1.21 | m | | |
| 10 | 1.89 | m | 32.5 | d | 1.94 | m | 32.4 | d | 1.89 | m | 32.5 | d | 1.89 | m | 32.3 | d |
| 11 | 1.87 | m | 54.6 | d | 1.89 | m | 55.0 | d | 1.87 | m | 55.0 | d | 1.85 | m | 55.0 | d |
| 12 | | | 83.5 | s | | | 83.3 | s | | | 83.7 | s | | | 83.2 | s |
| 13 | | | 221.9 | s | | | 221.9 | s | | | 221.2 | s | | | 221.7 | s |
| 14 | 3.32 | m | 40.4 | d | 3.17 | m | 43.0 | d | 3.69 | m | 39.8 | d | 3.15 | m | 42.6 | d |
| 15a | 2.07 | m | 35.9 | t | 2.20 | m | 31.5 | t | 3.50 | m | 42.0 | t | 2.08 | m | 29.9 | t |
| 15b | 1.31 | m | | | 1.41 | m | | | 2.68 | m | | | 1.27 | m | | |
| 16a | 3.59 | m | 60.3 | t | 2.65 | m | 24.3 | t | | | 189.3 | s | 2.35 | m | 23.7 | t |
| 16b | | | | | 2.62 | m | | | | | | | | | | |
| 17 | | | | | | | 151.5 | s | | | 149.2 | s | | | 109.2 | s |
| 18 | | | | | | | 114.5 | s | | | 130.5 | s | | | 149.8 | s |
| 19 | | | | | 7.31 | d (1.6) | 113.6 | d | 6.54 | (d, 1.8) | 116.6 | d | 3.15 | m | 38.1 | t |
| 20 | | | | | 6.20 | d (1.6) | 140.8 | d | 7.65 | (d, 1.8) | 145.6 | d | | | 173.6 | d |
| 21 | 1.66 | br s | 17.6 | q | 1.60 | br s | 17.7 | q | 1.68 | br s | 17.7 | q | 1.65 | br s | 17.7 | q |
| 22 | 0.98 | d (6.7) | 11.5 | q | 1.01 | d (6.8) | 11.6 | q | 0.98 | (d, 6.7) | 11.4 | q | 0.98 | (d, 6.7) | 11.5 | q |
| 23 | 0.75 | d (6.2) | 21.6 | q | 0.78 | d (6.0) | 21.7 | q | 0.76 | (d, 6.6) | 21.5 | q | 0.75 | (d, 6.2) | 21.6 | q |
| 24 | 1.04 | d (6.8) | 15.7 | q | 1.10 | d (6.8) | 15.1 | q | 1.11 | (d, 6.8) | 15.6 | q | 1.07 | (d, 6.7) | 14.9 | q |
| 25 | | | | | 1.98 | s | 9.9 | q | 2.35 | s | 11.7 | q | 1.75 | s | 10.8 | q |
| 4-OH | 4.49 | s | | | 4.71 | s | | | 4.52 | s | | | 4.66 | s | | |
| 5-OH | 4.24 | s | | | 4.26 | s | | | 4.25 | s | | | 4.27 | s | | |
| 12-OH | 3.87 | s | | | 3.92 | s | | | 3.95 | s | | | 3.92 | s | | |

TABLE 3
¹HNMR and ¹³CNMR data of compounds 9 to 12 (acetone-d₆, J in Hz)

| | 9 | | | | 10 | | | | 11 | | | | 12 | | | |
|---|---|---|---|---|---|---|---|---|---|---|---|---|---|---|---|---|
| No. | δ_{H} | | δ_{C} | | δ_{H} | | δ_{C} | | δ_{H} | | δ_{C} | | δ_{H} | | δ_{C} | |
| 1a | 4.04 | (d, 16.0) | 65.0 | t | 4.01 | (m) | 65.0 | t | 4.02 | br s | 66.7 | t | 4.00 | br s | 66.7 | t |
| 1b | 4.13 | (d, 16.0) | | | 4.16 | (m) | | | | | | | | | | |
| 2 | | | 137.3 | s | | | 136.9 | s | | | 138.7 | s | | | 138.0 | s |
| 3 | 4.96 | (m) | 122.7 | d | 4.96 | (m) | 122.8 | d | 5.72 | s | 121.7 | d | 5.72 | s | 122.7 | d |
| 4 | | | 73.9 | s | | | 73.9 | s | | | 71.7 | s | | | 71.5 | s |
| 5 | | | 97.6 | s | | | 97.6 | s | | | 100.6 | s | | | 100.6 | s |
| 6 | 1.92 | (m) | 42.6 | d | 1.92 | (m) | 42.6 | d | 1.82 | m | 37.6 | d | 1.82 | m | 37.5 | d |
| 7 | 1.58 | (m) | 44.0 | d | 1.60 | (m) | 44.0 | d | 1.75 | m | 44.4 | d | 1.77 | m | 44.6 | d |
| 8a | 1.20 | (m) | 28.5 | t | 1.18 | (m) | 28.6 | t | 1.73 | m | 28.2 | t | 1.74 | m | 28.2 | t |
| 8b | 1.77 | (m) | | | 1.78 | (m) | | | 1.14 | m | | | 1.13 | m | | |
| 9a | 1.22 | (m) | 33.8 | t | 1.23 | (m) | 33.8 | t | 2.24 | m | 33.0 | t | 1.88 | m | 34.0 | t |
| 9b | 1.87 | (m) | | | 1.79 | (m) | | | 1.34 | m | | | 1.22 | m | | |
| 10 | 1.89 | (m) | 32.3 | d | 1.90 | (m) | 32.2 | d | 1.91 | m | 33.4 | d | 1.91 | m | 33.3 | d |
| 11 | 1.86 | (m) | 54.9 | d | 1.85 | (m) | 55.1 | d | 1.52 | m | 54.7 | d | 1.48 | m | 54.8 | d |
| 12 | | | 83.4 | s | | | 83.2 | s | | | 86.1 | s | | | 85.6 | s |
| 13 | | | 221.2 | s | | | 222.1 | s | | | 215.9 | s | | | 217.1 | s |
| 14 | 3.06 | (m) | 43.2 | d | 3.06 | (m) | 43.3 | d | 3.76 | m | 42.0 | d | 3.88 | m | 40.7 | d |
| 15a | 1.10 | (m) | 26.2 | t | 1.62 | (m) | 35.7 | t | 1.90 | m | 34.0 | t | 1.76 | m | 33.3 | t |
| 15b | 1.82 | (m) | | | 1.81 | (m) | | | 1.22 | m | | | 1.56 | m | | |
| 16a | 1.78 | (m) | 34.2 | t | 1.01 | (m) | 26.8 | t | 2.13 | m | 54.3 | d | 2.24 | m | 54.0 | d |
| 16b | 2.06 | (m) | | | 1.88 | (m) | | | | | | | | | | |
| 17 | | | 111.4 | s | | | 114.9 | s | | | 208.5 | s | | | 208.8 | s |
| 18 | | | 166.0 | s | | | 141.4 | s | | | 142.0 | s | | | 141.8 | s |
| 19 | 6.05 | (m) | 121.0 | d | 5.73 | (d, 2.4) | 126.9 | d | 7.15 | m | 156.8 | d | 7.14 | br s | 157.4 | d |
| 20 | | | 170.1 | d | 5.34 | (br s) | 106.7 | d | 4.56 | m | 75.8 | d | 4.56 | m | 75.5 | d |
| 21 | 1.69 | (s) | 18.0 | q | 1.69 | (m) | 18.0 | q | 1.59 | s | 18.6 | q | 1.58 | s | 18.6 | q |
| 22 | 0.98 | (d, 6.8) | 11.5 | q | 0.99 | (d, 6.8) | 11.5 | q | 0.96 | d (6.6) | 12.3 | q | 0.96 | d (6.5) | 12.4 | q |
| 23 | 0.74 | (d, 6.3) | 21.7 | q | 0.76 | (m) | 21.8 | q | 0.77 | d (6.1) | 21.6 | q | 0.77 | d (6.0) | 21.4 | q |
| 24 | 1.04 | (d, 6.8) | 15.6 | q | 1.03 | (d, 6.9) | 15.8 | q | 1.15 | d (7.2) | 18.6 | q | 1.11 | d (6.9) | 16.5 | q |
| 25 | 2.04 | m | 12.7 | q | 1.69 | (m) | 11.5 | q | 1.71 | m | 10.1 | q | 1.70 | br s | 10.1 | q |
| 17-OCH₃ | 3.11 | (s) | 50.3 | q | 3.06 | (s) | 49.5 | q | | | | | | | | |
| 20-OCH₃ | | | | | 3.41 | (s) | 55.8 | q | | | | | | | | |
| 4-OH | | | | | 4.14 | (s) | | | 4.63 | s | | | 4.07 | s | | |
| 5-OH | 4.65 | (s) | | | 4.24 | (s) | | | 5.34 | s | | | 5.19 | s | | |
| 12-OH | 3.91 | (s) | | | 3.88 | (s) | | | 4.61 | s | | | 4.58 | s | | |
| 20-OH | | | | | | | | | 4.52 | m | | | 4.45 | d (6.4) | | |

### Example 2

Specifically, a preparation method of the Leucosceptrum canum extract L01 included the following steps:
(1) The overground parts of the Leucosceptrum canum were collected from Yunnan Province, shade-dried and crushed to 30 mesh size to obtain 2.0 kg of sample, and
the sample was soaked in 10 L of petroleum ether at room temperature and subjected to ultrasonic extraction three times, with each time for 30 min. The extract solution was combined after filtration;
(2) The above extract solution was subjected to concentration by a rotary evaporator at 45° C. to obtain 30 g of extractum;
(3) The above extractum was dissolved in 0.5 L of chloroform, mixed with 33 g of silica gel of 200 mesh size to 300 mesh size, allowed to air drying, ground and sieved, loaded with 240 g of the silica gel filler of 200 mesh size to 300 mesh size to allow column chromatography, elution was conducted with 100% petroleum ether and 100% acetone in sequence, and an obtained acetone eluate was subjected to concentration to obtain the Leucosceptrum canum extract L01 (18 g).

### Example 3

The compounds 1 to 12 of the present disclosure were tested for in vitro immunosuppressive activity:

CD3, CD28 monoclonal antibodies, and kit for IFN-γ detection were purchased from BD Bioscience, Cell Counting Kit-8 (CCK-8) was purchased from MeilunBio®, and RPMI-1640 medium and fetal bovine serum were purchased from Biological Industries.

Preparation of samples to be tested: the 12 compounds of Example 1 were dissolved in DMSO, respectively to prepare 20 mM stock solutions.

Splenocytes were aseptically isolated from 6-8 week old female C57BL/6 mice, and T cells were isolated using a nylon wool column method. The T cells were then inoculated (4×10⁵ cells/well) in a 96-well plate (which was coated with 5 μg/mL of CD3 monoclonal antibody). At the same time, a prepared compound solution of 40 μM, 20 μM, 10 μM, 5 μM, 2.5 μM, or 1.25 μM along with a 2 μg/mL solution of CD28 monoclonal antibody were added into each group, respectively, which were cultured in a 37° C./5% CO₂ incubator. After 48 h, the supernatant of the cells was collected and the IFN-γ secretion in the supernatant was detected by an Enzyme-Linked Immuno-Sorbent Assay (ELISA); 10 μL of CCK-8 solution was added into each well, and OD₄₅₀ was measured after incubation at 37° C. for 4 h to evaluate cell proliferation. In the experiment, a Cyclosporine A (CsA) positive control group, a DMSO negative control group, an unstimulated group, and a blank control group were also set up. The inhibitory activity of the compounds on IFN-γ was calculated, and the half-maximal inhibitory concentration (IC₅₀) was analyzed using GraphPadPrism software. The results were shown in Table 4.

TABLE 4
Inhibition secretion by compounds 1 to 12 of pro-
inflammatory cytokine IFN-γ by mouse T cells
stimulated by CD3/CD28 monoclonal antibodies

| Comp. | Inhibitory rate (%, 20 μM) | IC50 (μM) |
|---|---|---|
| 1 | 32.08 ± 5.46 | — |
| 2 | 51.28 ± 7.10 | — |
| 3 | 30.50 ± 6.55 | — |
| 4 | 26.74 ± 5.85 | — |
| 5 | 10.65 ± 2.74 | — |
| 6 | 66.85 ± 3.02 | 5.74 |
| 7 | 36.22 ± 8.27 | — |
| 8 | 43.13 ± 7.35 | — |
| 9 | 39.62 ± 7.93 | — |
| 10 | 10.39 ± 3.02 | — |
| 11 | 23.44 ± 1.24 | |
| 12 | 33.75 ± 2.27 | |
| CsA^{a} | 98.95 ± 0.32 | 1.08 × 10⁻³ |

^{a}CsA: by Cyclosporine A (CsA), a positive control.

As shown in Table 4, the compounds 1 to 12 significantly inhibited the secretion of the pro-inflammatory cytokine IFN-γ by mouse T cells stimulated by CD3/CD28 monoclonal antibodies, indicating that the type of compound had anti-inflammatory and immunosuppressive effects. Meanwhile, there was a certain structure-activity relationship between the type of compound and activity, providing a basis for future compound structure optimization and modification.

### Example 4

In Vivo Anti-Psoriasis Activity Test on the Leucosceptrum canum Extract L01:

BABL/6 mice were purchased from Beijing Vital River Experimental Animal Technology Co., Ltd., imiquimod cream was purchased from Sichuan Med Shine Pharmaceutical Co., Ltd., and Vaseline was purchased from Tianjin Zhiyuan Chemical Reagent Co., Ltd.

Establishment and pharmacodynamic study of a mouse model of imiquimod-induced psoriasis: the mice were randomly divided into a control group, a model group, a methotrexate (MTX) treatment (1 mg/kg) group, and Leucosceptrum canum sesterterpenoid treatment (50 mg/kg, 150 mg/kg, 450 mg/kg) groups, with 6 mice in each group. The mice were depilated on their backs before the experiment, and 62.5 mg of 0.5% imiquimod cream was evenly applied on the backs of mice while an equivalent amount of Vaseline was applied on the backs of mice in the normal control group from the 1st day, the application was performed once a day for 6 d. From the 1st day 1 to the 6th day, each treatment group was given the drug by gavage. During the experiment, the mice were weighed and scored for the Psoriasis Area and Severity Index (PASI) according to the following criteria: 0 points, normal skin without erythema and scales; 1 point, mild erythema, a few fine scales, and the skin at the lesion being slightly higher than normal skin; 2 points, moderate red plaques, the surface of the lesions being covered with scales, and the lesions being flaky or moderately raised; 3 points, severe dark red plaques, almost all the lesions being covered with thick scales or obvious lesions; 4 points, extremely severe and dark red plaques, all lesions being covered with highly thick layered scales, and the thickening and protrusion of the lesions being extremely obvious. After the experiment, the back skin of the mice was taken to make pathological sections. The levels of pro-inflammatory cytokines IFN-γ and IL-17A in mouse serum were detected by ELISA.

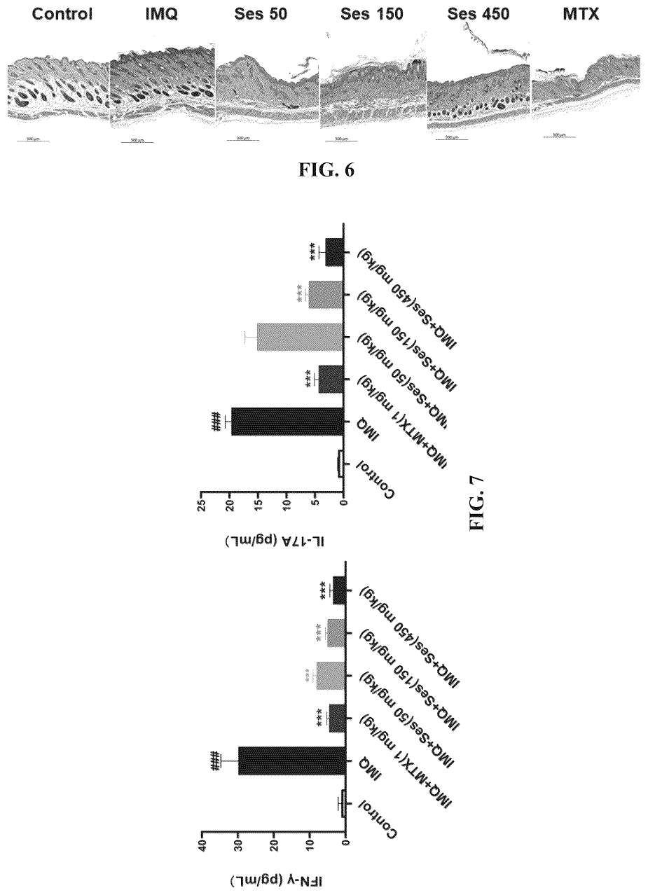

From FIG. 5, it can be seen that the Leucosceptrum canum extract L01 significantly improves the symptoms of psoriasis in mice. The experimental results of FIG. 6 indicate that the Leucosceptrum canum extract L01 significantly inhibits the pathological damage of the skin tissue of psoriasis mice. HE staining on the skin pathological sections reveals that psoriasis mice show thickening on the epidermis, parakeratosis and epidermal protrusions appear in the stratum corneum, thickening on the dermis, and lymphocyte infiltration, while the above symptoms of mice in the treatment groups were significantly improved. The experimental results in FIGS. 7A-7B indicate that the Leucosceptrum canum extract L01 significantly reduces the levels of pro-inflammatory cytokines IFN-γ and IL-17A in the serum of psoriasis mice. The above studies confirmed that the Leucosceptrum canum extract L01 had a desirable anti-psoriasis effect in vivo and showed a desirable application prospect in the clinical treatment of autoimmune diseases such as psoriasis.

### Example 5

### In Vivo Anti-Psoriasis Activity Test on the Sesterterpenoid Compound 6 in the Present Disclosure:

A method for establishing the imiquimod-induced psoriasis mouse model was the same as that in Example 4. The mice were randomly divided into a control group, a model group, a methotrexate (MTX) treatment group (1 mg/kg), and compound 6 treatment groups (25 and 50 mg/kg), with 6 mice in each group. From the 1st day to the 6th day, drugs were given to each treatment group by intraperitoneal injection. During the experiment, the mice were weighed and scored for PASI. After the experiment, the back skin of the mice was taken to make pathological sections. The levels of pro-inflammatory cytokines IFN-γ and IL-17A in mouse serum were detected by ELISA.

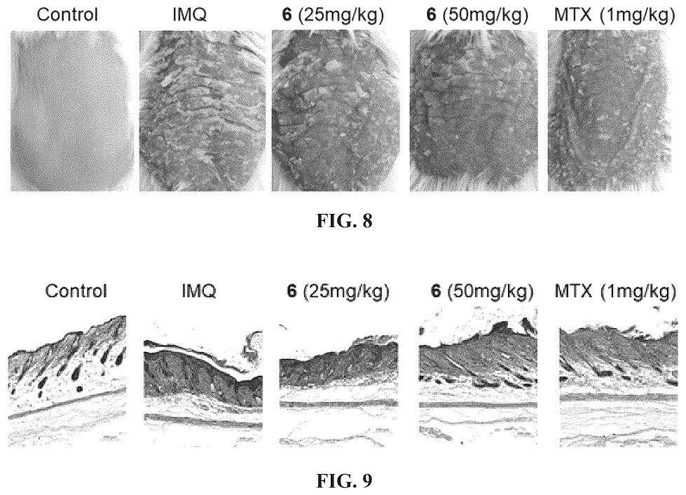

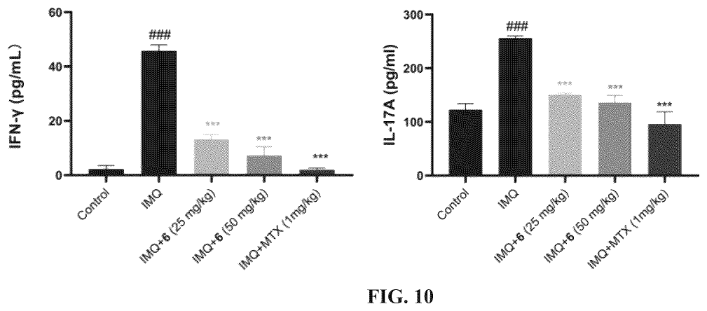

The experimental results in FIG. 8 show that compound 6 significantly improves the symptoms of psoriasis in mice. FIG. 9 shows that compound 6 significantly inhibits the pathological damage of the skin tissue of psoriasis mice. HE staining on the skin pathological sections reveals that psoriasis mice show thickening of the epidermis, parakeratosis and epidermal protrusions in the stratum corneum, thickening of the dermis, and lymphocyte infiltration, while the above symptoms of mice in the treatment group were significantly improved. The experimental results in FIGS. 10A-10B indicate that the compound 6 significantly reduces the levels of pro-inflammatory cytokines IFN-γ and IL-17A in the serum of psoriasis mice. The above studies indicated that the compound 6 had a desirable anti-psoriatic effect in vivo and could be used to treat autoimmune diseases such as psoriasis.

### Formulation Example

In the following formulation examples, conventional reagents were selected and the formulations were prepared according to existing conventional methods. This use example only embodied that the Leucosceptrum canum extract L01 and at least one of compounds 1 to 12 in the present disclosure could be prepared into different formulations, and did not specifically limit the specific reagents and operations:
(1) The Leucosceptrum canum extract L01 or compounds 1 to 12 were prepared into tablets:

Tablet ingredients were: 10 mg the Leucosceptrum canum extract L01 or one or a mixture of any two or more selected from the group consisting of compounds 1 to 12 as a raw material, 180 mg of lactose, 55 mg of starch, and 5 mg of magnesium stearate;

The preparation method included: the raw material, lactose, and starch were mixed, which was uniformly wet with propylene glycol, a resulting wet mixture was sieved and dried, sieved again, added with magnesium stearate, and then compressed into tablets, with each tablet weighing 250 mg and containing 10 mg of the raw material.
(2) The Leucosceptrum canum extract L01 or compounds 1 to 12 were prepared into capsules:

Capsule ingredients were: 10 mg the Leucosceptrum canum extract L01 or one or a mixture of any two or more selected from the group consisting of compounds 1 to 12 as a raw material, 187 mg of lactose, and 3 mg of magnesium stearate.

The preparation method included: the ingredients was mixed with the auxiliary agents, sieved, and an obtained mixture was filled into hard gelatin capsules, with each capsule weighing 200 mg and containing 10 mg of the raw material.
(3) The Leucosceptrum canum extract L01 or one or a mixture of any two or more selected from the group consisting of compounds 1 to 12 as a raw material was dissolved in sterile water for injection, stirred until the raw material was dissolved, filtered through a sterile suction funnel and sterile fine filtration, and then packaged into ampoules, freeze-dried at low temperature, and aseptically sealed to obtain a drug powder injection.
(4) The Leucosceptrum canum extract L01 or one or a mixture of any two or more selected from the group consisting of compounds 1 to 12 as a raw material was dissolved in DMSO, added with water for injection according to a conventional method, finely filtered, sealed and sterilized to prepare an injection solution, where a concentration of the injection solution was (0.5-5) mg/mL.
(5) The Leucosceptrum canum extract L01 or one or a mixture of any two or more selected from the group consisting of compounds 1 to 12 as a raw material was prepared into an oral liquid according to a conventional method for preparing an oral liquid.

The above are merely preferred implementations of the present application. It should be noted that a person of ordinary skill in the art may further make several improvements and modifications without departing from the principle of the present application, and such improvements and modifications should be deemed as falling within the protection scope of the present application.

1. A sesterterpenoid compound shown in any one of structural formulas 9 to 12 as follows:

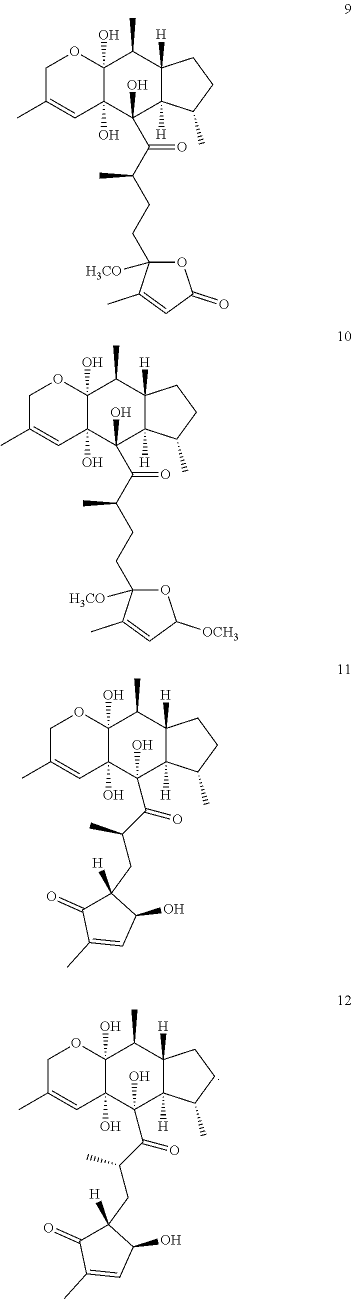

2. A method for preparing the sesterterpenoid compounds 9 to 12 according to claim 1, comprising the following steps:
extracting a Leucosceptrum canum with an organic solvent to obtain an extract solution; wherein the organic solvent is one or more selected from the group consisting of methanol, ethanol, acetone, chloroform, dichloromethane (DCM), and petroleum ether; the extraction is selected from the group consisting of cold soaking extraction and hot reflux extraction; the cold soaking extraction is conducted at room temperature 2 to 5 times for 24 h to 48 h each time; and the hot reflux extraction is conducted at 40° C. to 50° C. for 4 h to 6 h;
vacuum distilling the extract solution for concentration to obtain an extractum; wherein the vacuum distillation is conducted at 30° C. to 60° C.; and
dissolving the extractum, and conducting column chromatography separation and high-performance liquid chromatography (HPLC) separation in sequence to obtain the sesterterpenoid compounds; wherein the column chromatography comprises silica gel column chromatography, middle chromatogram isolated (MCI) column chromatography or reverse phase C₁₈ column chromatography, and dextran gel column chromatography; and the HPLC separation is conducted by preparative HPLC or semi-preparative HPLC;
the silica gel column chromatography has a silica gel filler of 200 mesh size to 300 mesh size; the silica gel column chromatography adopts a first gradient elution, and a mobile phase of the first gradient elution is a solvent mixture of a solvent A and a solvent B at a volume ratio ranging from 1:0 to 0:1; and the solvent A is any one selected from the group consisting of DCM, chloroform, and petroleum ether, and the solvent B is any one selected from the group consisting of DCM, chloroform, acetone, ethyl acetate, and methanol;
the MCI column chromatography or the reverse phase C₁₈ column chromatography adopts second gradient elution, and a mobile phase of the second gradient elution is a solvent mixture of methanol and water at a volume fraction of 50% to 100%;
a mobile phase of the dextran gel column chromatography is a solvent mixture of DCM and methanol or a solvent mixture of chloroform and methanol at a volume ratio of 1:1; and
chromatographic conditions of the HPLC separation comprise: a chromatographic column is selected from the group consisting of a C₁₈ chromatographic column, a C₈ chromatographic column, a phenyl chromatographic column, and a silica gel chromatographic column, and the chromatographic column has a column temperature of 20° C. to 50° C.; gradient elution is conducted with a mobile phase being a solvent mixture of acetonitrile and water or a solvent mixture of methanol and water at a volume ratio of 100:0 to 5:95; and a flow rate is 1 mL/min to 3 mL/min.

3. The method according to claim 2, wherein the Leucosceptrum canum is selected from the group consisting of an overground part and any tissue of Leucosceptrum canum.

4.-9. (canceled)

10. The method according to claim 2, wherein the first gradient elution of the silica gel column chromatography in the step (3) comprises the following steps:
conducting gradient elution with a mixture of petroleum ether and DCM at volume ratios of 1:0, 1:1, and 0:1 and then with a mixture of DCM and acetone at volume ratios of 9:1, 4:1, and 1:1 in sequence, subjecting obtained fractions to vacuum concentration, conducting detection by thin layer chromatography (TLC), and combining same fractions to obtain six fractions to be separated, namely a fraction Fr.1 from petroleum ether, a fraction Fr.2 from petroleum ether:DCM=1:1, a fraction Fr.3 from DCM, a fraction Fr.4 from DCM:acetone=9:1, a fraction Fr.5 from DCM:acetone=4:1, and a fraction Fr.6 from DCM:acetone=1:1;
subjecting the fraction Fr.4 to the MCI column chromatography or the reverse phase C₁₈ column chromatography, and then subjecting an obtained fraction Fr.4-2 to the dextran gel column chromatography to obtain four sub-fractions, namely, Fr.4-2-1, Fr.4-2-2, Fr.4-2-3, and Fr.4-2-4; and
subjecting the sub-fractions Fr.4-2-1 to Fr.4-2-4 to the HPLC separation using an Agilent 1200 semi-preparative liquid chromatograph with a ZorbaxSB-C₁₈ column, 5 μm, 9.4×250 mm, 3 mL/min, and a column temperature of 35° C.; wherein compounds 3, 4, and 6 are obtained from the Fr.4-2-1 under an elution condition of MeCN and H₂O at a volume ratio (v/v) of 75:25; compounds 1, 8, and 10 are obtained from the Fr.4-2-2 under an elution condition of MeCN and H₂O at a v/v of 70:30; compounds 7, 9, and 12 are obtained from the Fr.4-2-3 under an elution condition of MeCN and H₂O at a v/v of 80:20; and compounds 11, 2, and 5 are obtained from the Fr.4-2-4 under an elution condition of MeCN and H₂O at a v/v of 60:40; and
the compounds 1 to 8 have structural formulas as follows:

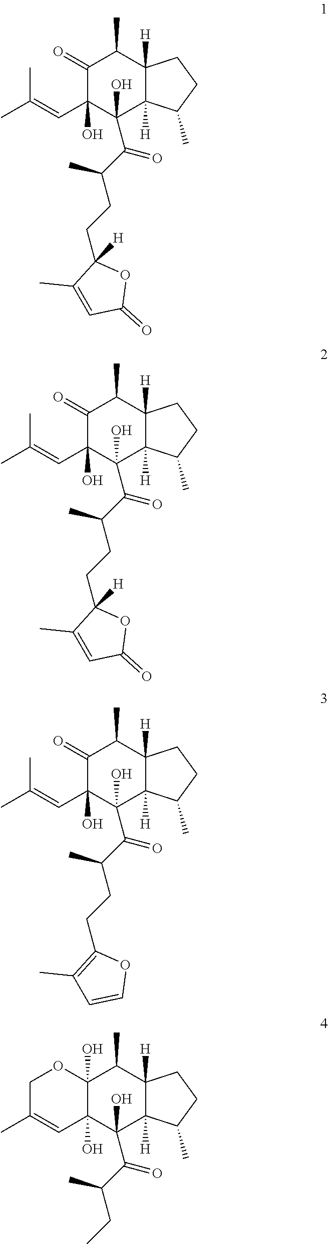

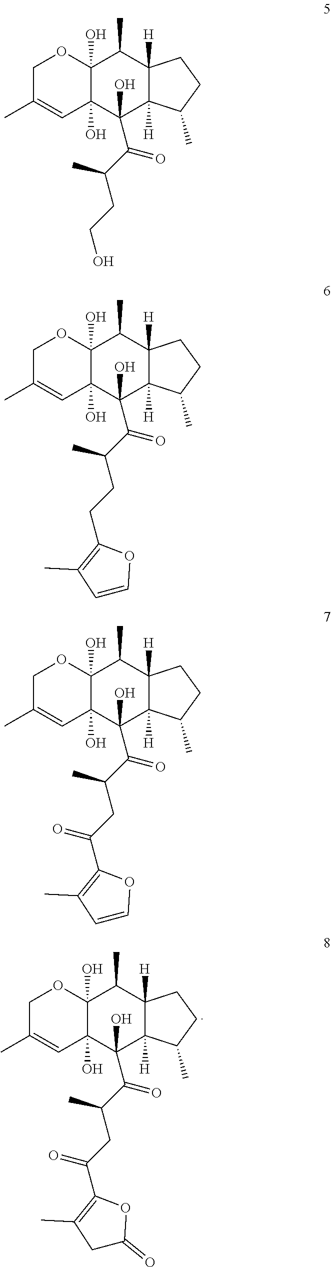

11. The method according to claim 10, wherein
subjecting the fraction Fr.4 to MCI column chromatography, and conducting gradient elution with methanol and water at volume fractions of 50%, 60%, 70%, 80%, 90%, and 100% to obtain four sub-fractions Fr.4-1 to Fr.4-4.

12. The method according to claim 10, wherein
subjecting the fraction Fr.4-2 to the dextran gel column chromatography and then eluting with DCM and methanol at a volume ratio of 1:1 to obtain four sub-fractions Fr.4-2-1 to Fr.4-2-4.

13. A Leucosceptrum canum extract L01 prepared by a method comprising the following steps:
(1) mixing a crushed Leucosceptrum canum with an organic solvent to allow an ultrasonic treatment, and collecting a resulted extract solution; wherein the organic solvent is one or more selected from the group consisting of methanol, ethanol, acetone, chloroform, DCM, and petroleum ether; a solid-liquid ratio of an overground part of the crushed Leucosceptrum canum and the organic solvent is 1:3 to 1:8; and a time for the ultrasonic treatment is 10 min to 40 min;
(2) subjecting the extract solution to concentration to obtain a crude extract; wherein the concentration is conducted by rotary evaporation at 30° C. to 60° C.; and
(3) subjecting the crude extract to elution by silica gel column chromatography, collecting a resulting acetone elution phase, and conducting concentration to obtain the Leucosceptrum canum extract L01; wherein eluents for the elution by silica gel column chromatography are petroleum ether and acetone in sequence; and the concentration is conducted by rotary evaporation at 30° C. to 60° C.

14. A method for preparing an anti-inflammatory drug or an immunosuppressive drug, comprising using at least one selected from the group consisting of sesterterpenoid compounds 1 to 12, wherein the sesterterpenoid compounds 1 to 12 are shown in structure formulas 1 to 12 as follows:

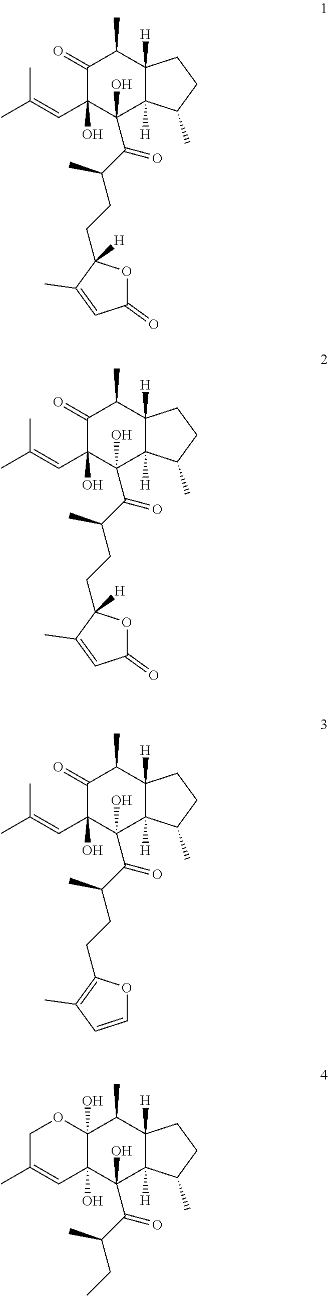

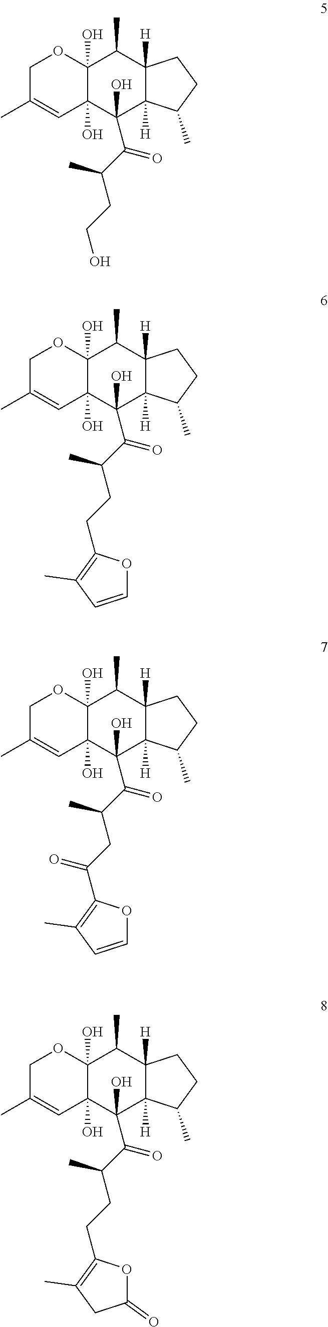

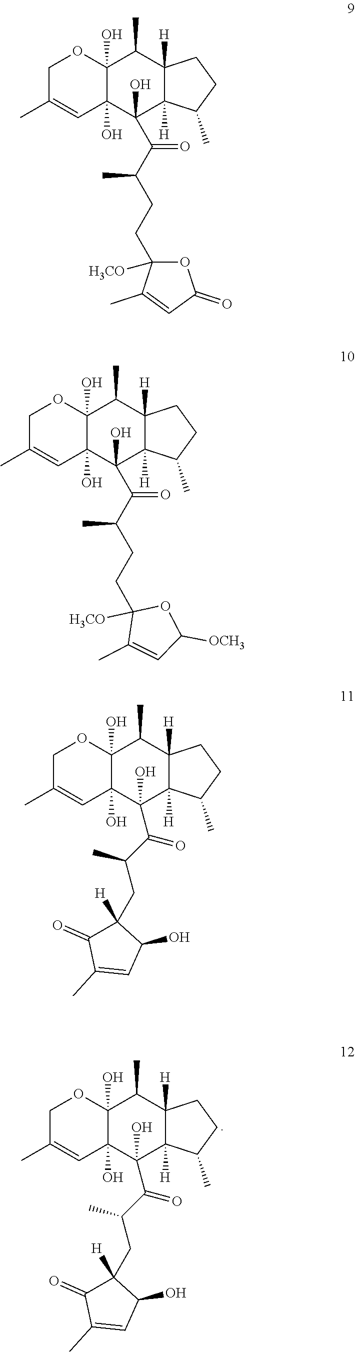

15. A method for preparing a pro-inflammatory cytokine inhibitor or an anti-psoriasis drug, comprising using any one or more selected from the group consisting of sesterterpenoid compounds 1 to 12 according to claim 14.

16. A method for preparing an anti-inflammatory drug or an immunosuppressive drug, comprising using the Leucosceptrum canum extract L01 according to claim 13.

17. A method for preparing a pro-inflammatory cytokine inhibitor or an anti-psoriasis drug, comprising using the Leucosceptrum canum extract L01 according to claim 13.

18. A pharmaceutical composition, comprising a pharmaceutically acceptable carrier and at least one selected from the group consisting of the sesterterpenoid compounds 1 to 12 according to claim 14.

19. A pharmaceutical composition, comprising a pharmaceutically acceptable carrier and the Leucosceptrum canum extract L01 according to claim 13.

20. A method for preparing the Leucosceptrum canum extract L01 according to claim 13, comprising the following steps:
(1) collecting an overground part of Leucosceptrum canum to allow shade drying, crushing to 30 mesh size, fully mixing a crushed Leucosceptrum canum with 10 L of petroleum ether at room temperature, subjecting a resulting mixture to ultrasonic extraction three times for 30 min each time, conducting filtration, and combining obtained extract solutions;
(2) subjecting the obtained extract solution to concentration by a rotary evaporator at 45° C. to obtain an extractum; and
(3) dissolving the extractum in chloroform, mixing with a silica gel of 200 mesh size to 300 mesh size, conducting air drying, grinding and sieving, loading with the silica gel filler of 200 mesh size to 300 mesh size to allow column chromatography, conducting elution with petroleum ether and acetone in sequence, and subjecting an obtained acetone eluate to concentration to obtain the Leucosceptrum canum extract L01.

21. The pharmaceutical composition according to claim 18, wherein a dosage form of the pharmaceutical composition is selected from the group consisting of a liquid preparation, a solid preparation, a spray, and an aerosol.

22. The pharmaceutical composition according to claim 21, wherein the liquid preparation is selected from the group consisting of an injection, a suspension, an emulsion, a solution, and a syrup; and the solid preparation is selected from the group consisting of a tablet, a capsule, a granule, and an instant granule.

23. A tablet, comprising the following ingredients: 10 mg of Leucosceptrum canum extract L01 or one or a mixture of any two or more selected from the group consisting of sesterterpenoid compounds 1 to 12, 180 mg of lactose, 55 mg of starch, and 5 mg of magnesium stearate; wherein
the Leucosceptrum canum extract L01 is the Leucosceptrum canum extract L01 according to claim 13; and
the sesterterpenoid compounds 1 to 12 are shown in structure formulas 1 to 12 as follows:

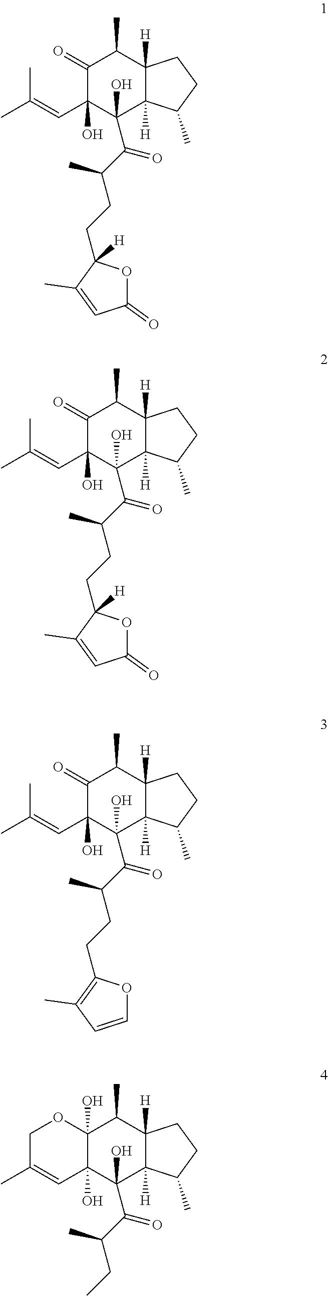

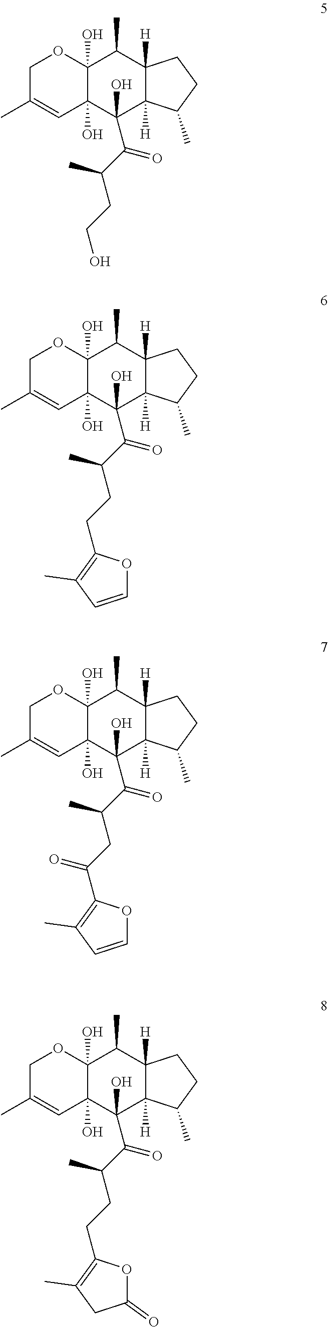

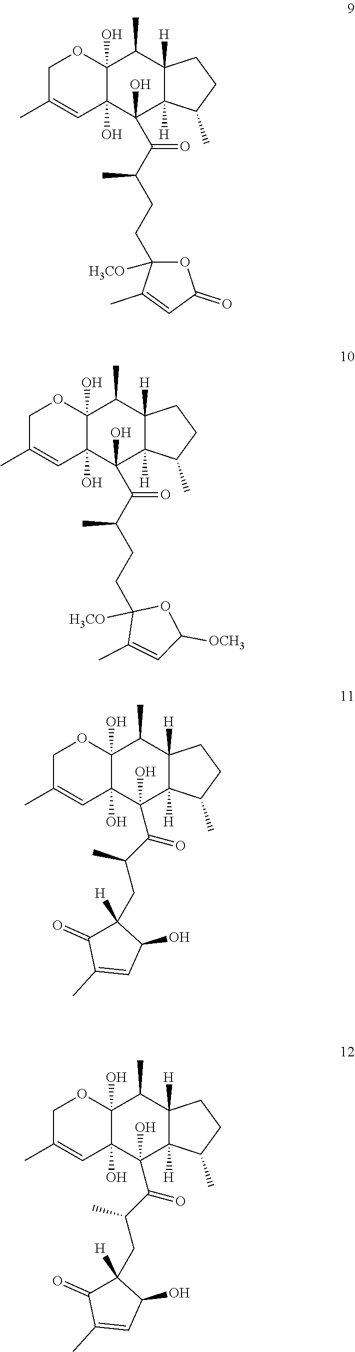

24. A capsule, comprising the following ingredients: 10 mg of Leucosceptrum canum extract L01 or one or a mixture of any two or more selected from the group consisting of sesterterpenoid compounds 1 to 12 as a raw material, 187 mg of lactose, and 3 mg of magnesium stearate; wherein
the Leucosceptrum canum extract L01 is the Leucosceptrum canum extract L01 according to claim 13, and
the sesterterpenoid compounds 1 to 12 are shown in structure formulas 1 to 12 as follows;

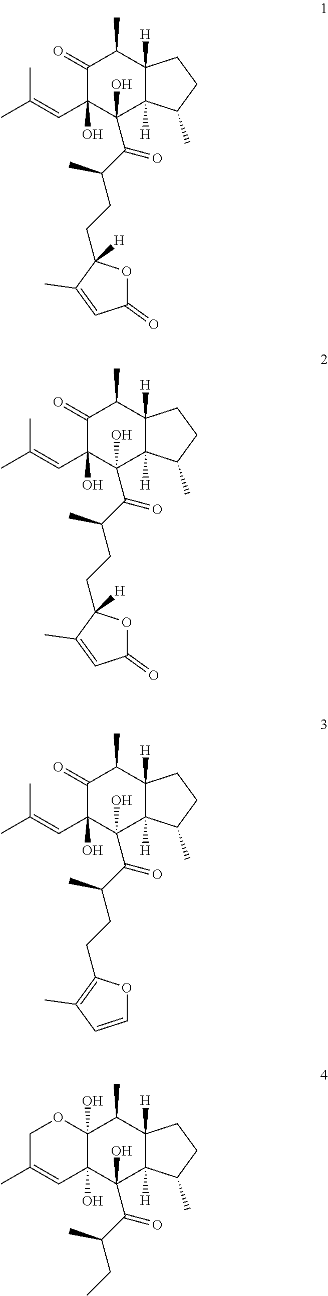

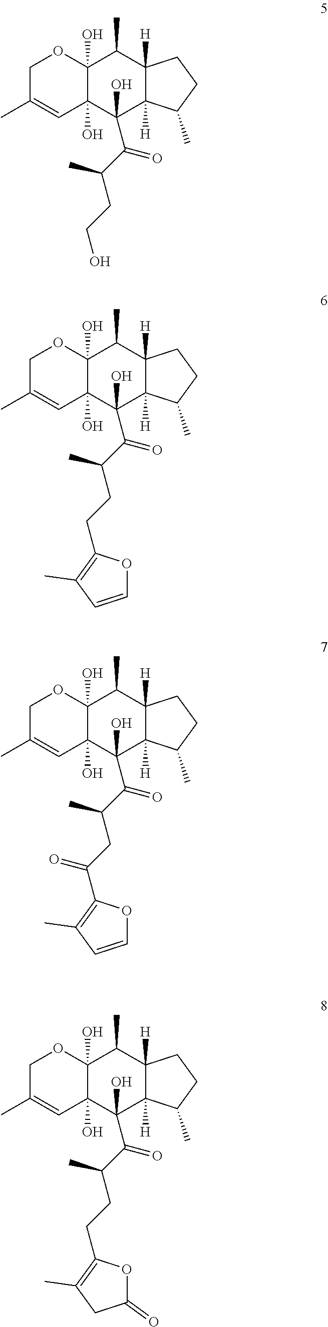

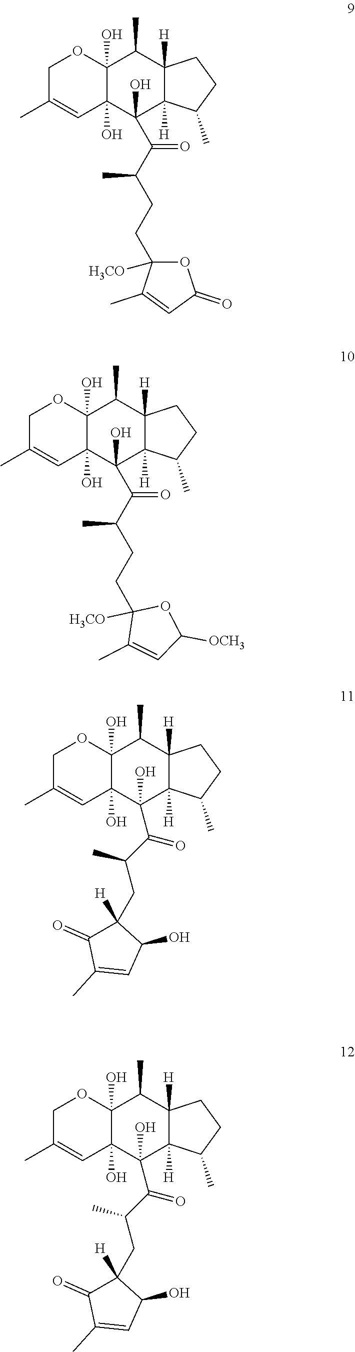

25. The pharmaceutical composition according to claim 19, wherein a dosage form of the pharmaceutical composition is one selected from the group consisting of a liquid preparation, a solid preparation, a spray, and an aerosol.

26. The pharmaceutical composition according to claim 25, wherein the liquid preparation is one selected from the group consisting of an injection, a suspension, an emulsion, a solution, and a syrup; and the solid preparation is one selected from the group consisting of a tablet, a capsule, a granule, and an instant granule.
